(19) United States
(12) Patent Application Publication
(10) Pub. No.: US 2025/0050008 A1
(43) Pub. Date: Feb. 13, 2025

(54) TEMPERATURE DETECTION AND PROTECTION FOR NEGATIVE PRESSURE WOUND THERAPY SYSTEMS

(71) Applicant: T.J.Smith and Nephew,Limited, Hull (GB)

(72) Inventors: Allan Kenneth Frazer Grugeon Hunt, Beverley (GB); Felix Clarence Quintanar, Hull (GB); Kirsty Margaret Strachan, Hull (GB)

(21) Appl. No.: 18/720,424

(22) PCT Filed: Jan. 6, 2023
(86) PCT No.: PCT/EP2023/050237

§ 371 Date: Jun. 14, 2024

(30) Foreign Application Priority Data
Jan. 10, 2022 (GB) 2200209.1

(51) Int. Cl. A61M 1/00; A61F 13/0203

(52) U.S. Cl. CPC A61M 1/962; A61F 13/0203

(57) ABSTRACT

A negative pressure wound therapy system can include a negative pressure source configured to provide negative pressure to a wound covered by a wound dressing, an electronic control circuitry configured to operate the pressure source, a power source configured to provide power to the pressure source and the electronic control circuitry, and an integrated circuit configured to receive power from the power source, the integrated circuit including an electronic protection circuitry configured to monitor a temperature of the power source, responsive to the temperature satisfying at least one temperature threshold, cause the electronic control circuitry to adjust operation of the pressure source or prevent supply of power, and responsive detection of at least one of a reverse current condition or an excessive current condition, prevent supply of power. The pressure source, electronic control circuitry, power source, and integrated circuit can be disposed on or within the wound dressing.

### TECHNICAL FIELD

Embodiments described herein relate to apparatuses, systems, and methods the treatment of wounds, for example using dressings in combination with negative pressure wound therapy.

### DESCRIPTION OF THE RELATED ART

The treatment of open or chronic wounds that are too large to spontaneously close or otherwise fail to heal by means of applying negative pressure to the site of the wound is well known in the art. Negative pressure wound therapy (“NPWT”) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

### SUMMARY

A negative pressure wound therapy system can include a wound dressing configured to be placed over a wound, the wound dressing configured to absorb fluid aspirated from the wound. The system can include a negative pressure source disposed on or within the wound dressing, the negative pressure source configured to aspirate fluid from the wound. The system can include an electronic control circuitry disposed on or within the wound dressing and configured to operate the negative pressure source. The system can include a power source disposed on or within the wound dressing and configured to provide power to the negative pressure source and the electronic control circuitry. The system can include an integrated circuit disposed on or within the wound dressing and configured to receive power from the power source. The integrated circuit can include an electronic protection circuitry configured to monitor a temperature of the power source. The electronic processing circuitry can be configured to, responsive to the temperature satisfying at least one temperature threshold, cause the electronic control circuitry to adjust operation of the negative pressure source or prevent supply of power from the power source. The electronic processing circuitry can be configured to, responsive detection of at least one of a reverse current condition or an excessive current condition, prevent supply of power from the power source.

The negative pressure wound therapy system of any of the preceding paragraphs and/or any of the apparatuses, systems, or devices disclosed herein can include one or more of the following features. The electronic protection circuitry can include a switch configured to be opened to prevent supply of power from the power source. The integrated circuit can be disposed between the power source and the negative pressure source. The integrated circuit can include a temperature sensor configured to measure the temperature of the power source. The system can include a temperature sensor configured to measure the temperature of the power source and communicate the measured temperature to the electronic protection circuitry.

The negative pressure wound therapy system of any of the preceding paragraphs and/or any of the apparatuses, systems, or devices disclosed herein can include one or more of the following features. The electronic protection circuitry can be configured to monitor a current provided by the power source. The electronic protection circuitry can be configured to, responsive to the temperature satisfying the at least one temperature threshold and the current not increasing over a duration of time, cause the electronic control circuitry to deactivate the negative pressure source. The electronic protection circuitry can be configured to, responsive to the temperature satisfying the at least one temperature threshold and the current increasing over the duration of time, prevent supply of power from the power source. The at least one temperature threshold can include first and second temperature thresholds. The second temperature threshold can be associated with temperature that is higher than temperature associated with the first temperature threshold. The integrated circuit can be configured to, responsive to the temperature satisfying the first temperature threshold and the current not increasing over the duration of time, cause the electronic control circuitry to decrease activity of the negative pressure source. The integrated circuit can be configured to, responsive to the temperature satisfying the second temperature threshold and the current not increasing over the duration of time, cause the electronic control circuitry to deactivate the negative pressure source. The electronic protection circuitry can be configured to, responsive to the temperature not satisfying the at least one temperature threshold and the current increasing over the duration of time, cause the electronic control circuitry to deactivate the negative pressure source. The integrated circuit can be configured to determine that the current is increasing responsive to the current satisfying a maximum current threshold. The maximum current threshold can be 320 mA.

A negative pressure wound therapy system can include a wound dressing configured to be placed over a wound, the wound dressing configured to absorb fluid aspirated from the wound. The system can include a plurality of electronic components disposed on or within the wound dressing. The plurality of electronic components can include a negative pressure source disposed on or within the wound dressing, the negative pressure source configured to aspirate fluid from the wound. The plurality of electronic components can include a power source configured to provide power to the negative pressure source. The system can include a temperature protection system disposed on or within the wound dressing. The temperature protection system can include at least first and second substances enclosed in a pouch. The temperature protection system can include a trigger configured to facilitate at least one of mixing of or reaction between the first and second substances responsive to a temperature of at least one electronic component of the plurality of electronic components satisfying a temperature threshold indicative of excessive temperature and trigger an endothermic reaction that limits an amount of heat generated by the at least one electronic component.

The negative pressure wound therapy system of any of the preceding paragraphs and/or any of the apparatuses, systems, or devices disclosed herein can include one or more of the following features. The trigger can be a bimetallic switch. The bimetallic switch can be a bimetallic disk configured to change shape responsive to a temperature of the bimetallic disk satisfying the temperature threshold. The pouch can be positioned between the at least one electronic component and the wound. The pouch can at least partially surround the at least one electronic component. The at least one electronic component can be the power source. The at least one electronic component can be the negative pressure source.

The negative pressure wound therapy system of any of the preceding paragraphs and/or any of the apparatuses, systems, or devices disclosed herein can include one or more of the following features. The first and second substances can be separated from one another. The first substance can be stored in a first section of the pouch and the second substance is stored in a second section of the pouch. The trigger can be configured to cause a breach in a separation between the first and second sections responsive to the temperature of the at least one electronic component satisfying the temperature threshold. The first substance can be a salt or alcohol, and the second substance can be water. The first substance can be a urea salt or sodium chloride. The first substance can be propylene glycol.

Disclosed herein are methods of operating a negative pressure wound therapy system of any of the preceding paragraphs and/or any of the devices, apparatuses, or systems disclosed herein.

Disclosed herein is a wearable electronic device, system, or apparatus incorporating at least one of the integrated circuit or the temperature protection of any of the preceding paragraphs and/or any of the devices, systems, or devices disclosed herein. Disclosed herein are method of operating such wearable electronic devices, systems, or apparatuses.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIGS. 1A-1C illustrate a wound dressing incorporating a source of negative pressure and/or other electronic components within the wound dressing; FIGS. 2A-2B illustrate an electronics unit that may be incorporated into a wound dressing;

FIG. 3 is an exploded perspective view of an electronics assembly enclosing an electronics unit within a housing;

FIG. 4A illustrates a bottom perspective view of the electronics assembly of FIG. 3;

FIG. 4B illustrates a top perspective view of the electronics assembly of FIG. 3;

FIG. 5A is an exploded view of a wound dressing incorporating an electronics assembly within the wound dressing layers;

FIG. 5B illustrates a cross sectional layout of the material layers of a wound dressing incorporating an electronics assembly within the dressing;

FIGS. 6A-6B and 7A-7B illustrate components of an electronics assembly;

FIG. 8 a pump exhaust mechanism;

FIG. 9 illustrates a block diagram of the electronics of a TNP system;

FIG. 10 illustrates a block diagram of a protection circuitry for a TNP system;

FIG. 11 illustrates a switch utilized by the protection circuitry;

FIG. 12 illustrates reverse polarity protection;

FIG. 13 illustrates a block diagram of a protection circuitry for a TNP system;

FIGS. 14, 15A-15B, 16A-16C, 17A-17C, and 18A-18B illustrate a TNP system with endothermic protection.

### DETAILED DESCRIPTION

Embodiments disclosed herein relate to apparatuses and methods of treating a wound with reduced pressure, including a source of negative pressure and wound dressing components and apparatuses. These apparatuses and components, including but not limited to wound overlays, backing layers, cover layers, drapes, sealing layers, spacer layers, absorbent layers, transmission layers, wound contact layers, packing materials, fillers and/or fluidic connectors are sometimes collectively referred to herein as dressings.

It will be appreciated that throughout this specification reference is made to a wound. It is to be understood that the term wound is to be broadly construed and encompasses open and closed wounds in which skin may be torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

It will be understood that embodiments of the present disclosure are generally applicable to use in NPWT or topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, 1013.25 mbar, etc.). Accordingly, a negative pressure value of −X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (such as, −40 mmHg is less than−60 mmHg). Negative pressure that is “more” or “greater” than −X mmHg corresponds to pressure that is further from atmospheric pressure (such as, −80 mmHg is more than −60 mmHg). In some cases, local ambient atmospheric pressure is used as a reference point, and such local atmospheric pressure may not necessarily be, for example, 760 mmHg.

The negative pressure range can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the negative pressure apparatus.

### Wound Dressing

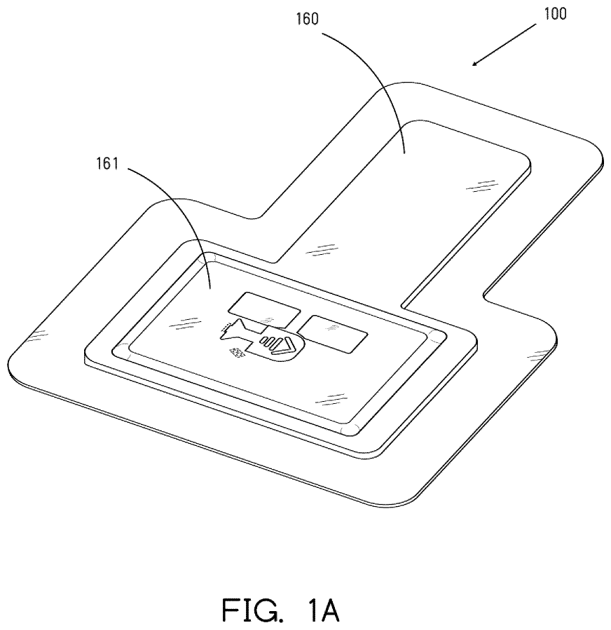

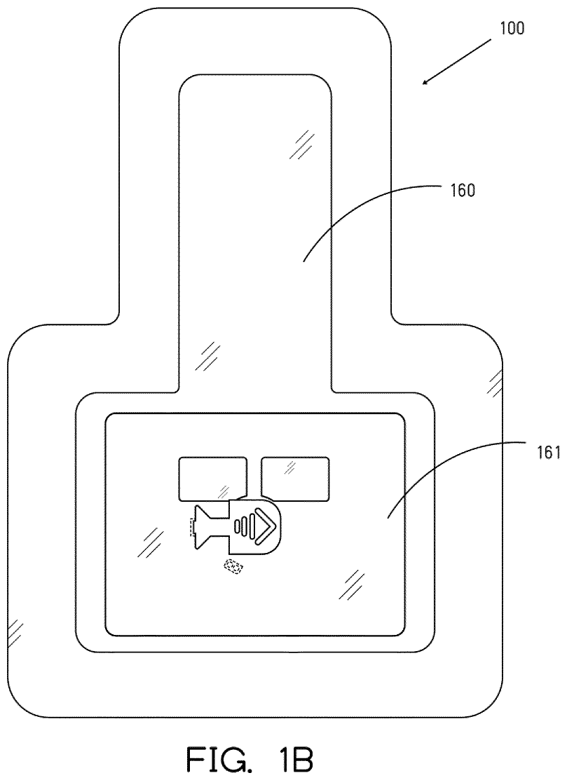

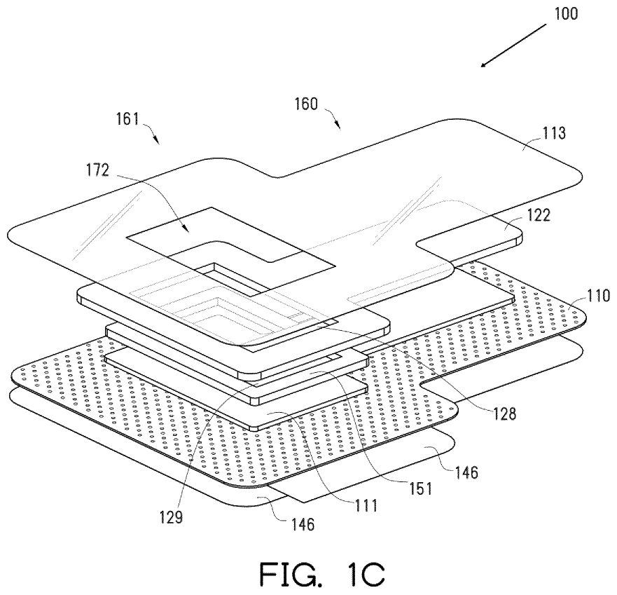

A source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. The material layers can include a wound contact layer, one or more absorbent layers, one or more transmission or spacer layers, and a backing layer or cover layer covering the one or more absorbent and transmission or spacer layers. The wound dressing can be placed over a wound and sealed to the wound with the pump and/or other electronic components contained under the cover layer within the wound dressing. The dressing can be provided as a single article with all wound dressing elements (including the pump) pre-attached and integrated into a single unit. A periphery of the wound contact layer can be attached to the periphery of the cover layer enclosing all wound dressing elements as illustrated in FIG. 1A-1C.

The pump and/or other electronic components can be configured to be positioned adjacent to or next to the absorbent and/or transmission layers so that the pump and/or other electronic components are still part of a single article to be applied to a patient. The pump and/or other electronics can be positioned away from the wound site. Although certain features disclosed herein may be described as relating to systems and method for controlling operation of a negative pressure wound therapy system in which the pump and/or other electronic components are positioned in or on the wound dressing, the systems and methods disclosed herein are applicable to any negative pressure wound therapy system or any medical device. FIGS. 1A-1C illustrate a wound dressing incorporating the source of negative pressure and/or other electronic components within the wound dressing. FIGS. 1A-1C illustrate a wound dressing 100 with the pump and/or other electronics positioned away from the wound site. The wound dressing can include an electronics area 161 and an absorbent area 160. The dressing can comprise a wound contact layer 110 (not shown in FIGS. 1A-1B) and a moisture vapor permeable film, cover layer or backing layer 113 positioned above the contact layer and other layers of the dressing. The wound dressing layers and components of the electronics area as well as the absorbent area can be covered by one continuous cover layer 113 as shown in FIGS. 1A-1C.

A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. In particular, the transmission layer 111 preferably ensures that an open air channel can be maintained to communicate negative pressure over the wound area even when the absorbent layer has absorbed substantial amounts of exudates. The layer 111 should preferably remain open under the typical pressures that will be applied during negative pressure wound therapy as described above, so that the whole wound site sees an equalized negative pressure. The layer 111 may be formed of a material having a three dimensional structure. For example, a knitted or woven spacer fabric (for example Baltex 7970 weft knitted polyester) or a non-woven fabric could be used.

Further, one or more absorbent layers (such as layers 122, 151) for absorbing and retaining exudate aspirated from the wound can be utilized. A superabsorbent material can be used in the absorbent layers 122, 151. The one or more layers 122, 151 of absorbent material may be provided above the transmission layer 111. Since in use each of the absorbent layers experiences negative pressures, the material of the absorbent layer can be chosen to absorb liquid under such circumstances. The absorbent layers 122. 151 may comprise a composite comprising superabsorbent powder, fibrous material such as cellulose, and bonding fibers. The composite can be an airlaid, thermally-bonded composite.

The electronics area 161 can include a source of negative pressure (such as a pump) and some or all other components of the TNP system, such as power source(s), sensor(s), connector(s), user interface component(s) (such as button(s), switch(es), speaker(s), screen(s), etc.) and the like, that can be integral with the wound dressing. For example, the electronics area 161 can include a button or switch (shown in FIGS. 1A-1B as being covered by a pull tab). The button or switch can be used for operating the pump (such as, turning the pump on/off).

The electronics area 161 of the dressing can comprise one or more layers of transmission or spacer material and/or absorbent material and electronic components can be embedded within the one or more layers of transmission or spacer material and/or absorbent material. The layers of transmission or absorbent material can have recesses or cut outs to embed the electronic components within whilst providing structure to prevent collapse. As shown in FIG. 1C, recesses 128 and 129 can be provided in absorbent layers 151 and 122, respectively.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

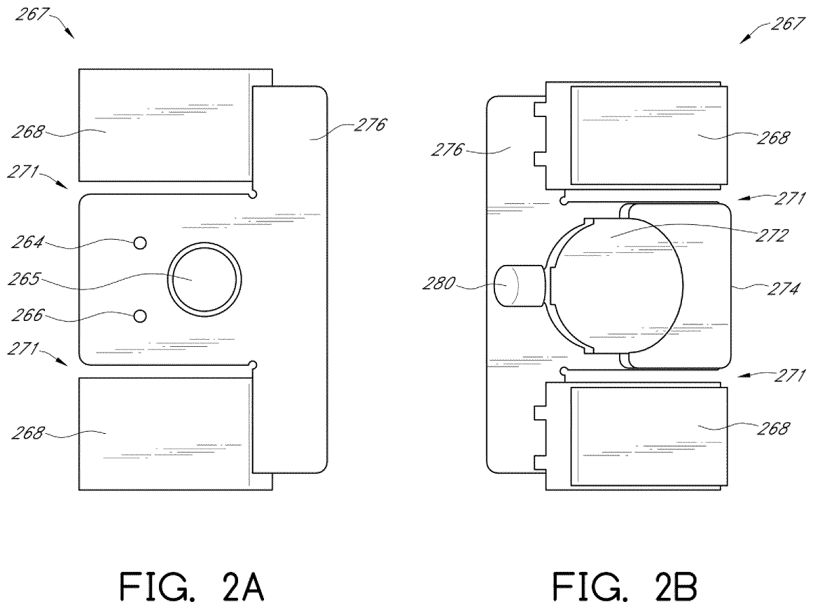

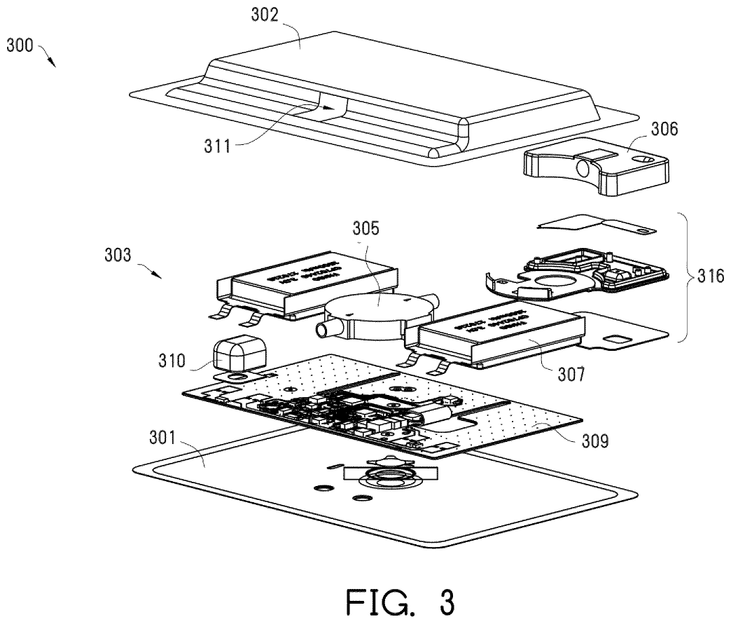

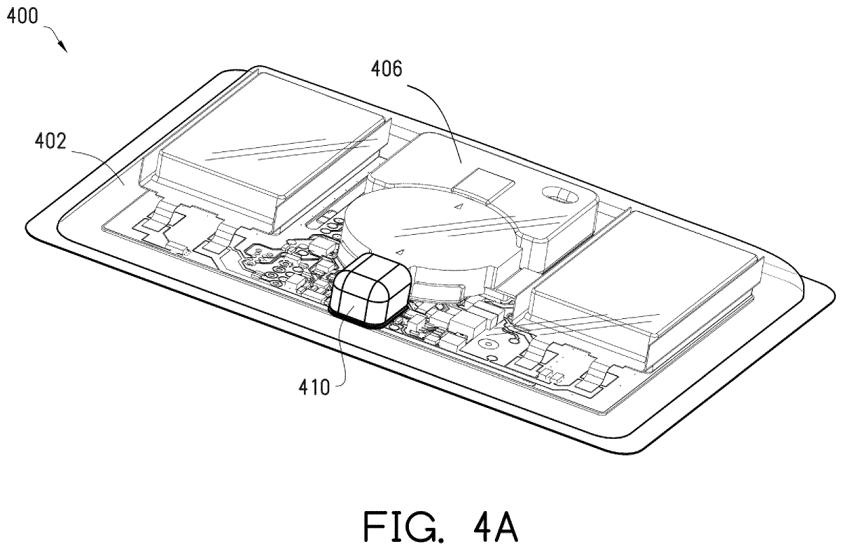

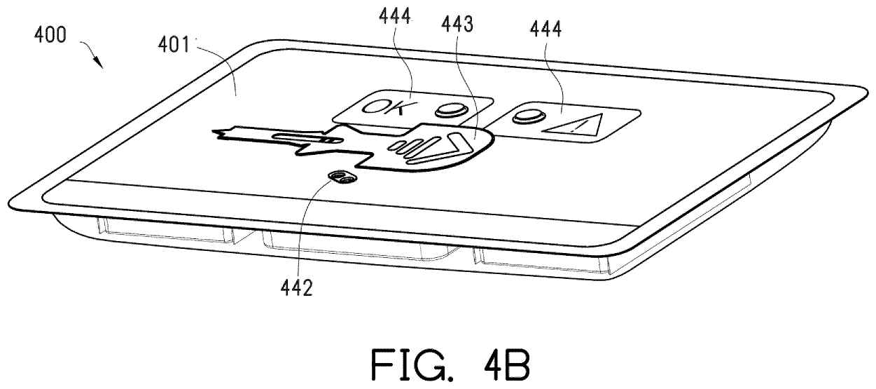

The cover layer may include a cutout 172 positioned over at least a portion of the aperture 128 in the absorbent layer 122 to allow access and fluid communication to at least a portion of the absorbent layers 122 and 151, transmission layer 111, and would contact layer 110 positioned below. An electronics assembly such as described below can be positioned in the apertures 128, 129, and 172 of the first and second absorbent material 151 and 122 and the cover layer 113. The electronics assembly can include a pump, power source, and a printed circuit board as described with reference to FIGS. 3 and 4A-4B.

Before use, the dressing can include one or more delivery layers 146 adhered to the bottom surface of the wound contact layer. The delivery layer 146 can cover adhesive or apertures on the bottom surface of the wound contact layer 110. The delivery layer 146 can provided support for the dressing and can assist in sterile and appropriate placement of the dressing over the wound and skin of the patient. The delivery layer 146 can include handles that can be used by the user to separate the delivery layer 146 from the wound contact layer 110 before applying the dressing to a wound and skin of a patient.

### Electronics Assembly Incorporated Within the Wound Dressing

FIGS. 2A-2B illustrate an electronics unit 267 that can be incorporated into a wound dressing. FIG. 2A illustrates the top view of the electronics unit. FIG. 2B illustrates a bottom or wound facing surface of the electronics unit. The electronics unit 267 can include a pump 272 and one or more power sources 268, such as batteries. The electronics unit 267 can include a circuit board 276 configured to be in electrical communication with the pump 272 and/or power source 268. The circuit board 276 can be flexible or substantially flexible.

As illustrated in FIG. 2A, the electronics unit 267 can include single button or switch 265 on the upper surface of the unit. The single button or switch 265 can be used as an on/off button or switch to stop and start operation of the pump and/or electronic components. The electronics unit 267 can also include one or more vents or exhaust apertures 264 on the circuit board 276 for expelling the air exhausted from the pump. As shown in FIG. 2B, a pump outlet exhaust mechanism 274 (sometimes referred to as pump exhaust mechanism or pump outlet mechanism) can be attached to the outlet of the pump 272.

The electronics unit 267 can include a pump inlet protection mechanism 280 as shown in FIG. 2B positioned on the portion of the electronics unit closest to the absorbent area and aligned with the inlet of the pump 272. The pump inlet protection mechanism 280 is positioned between the pump inlet and the absorbent area or absorbent layer of the dressing. The pump inlet protection mechanism 280 can include hydrophobic material to prevent fluid from entering the pump 272. The pump inlet protection mechanism 280 (or any of the inlet protection mechanisms disclosed herein) can include a filter.

The upper surface of the electronics unit 267 can include one or more indicators 266 for indicating a condition of the pump and/or level of pressure within the dressing. The indicators can be small LED lights or other light source that are visible through the dressing components or through holes in the dressing components above the indicators. The indicators can be green, yellow, red, orange, or any other color. For example, there can be two lights, one green light and one orange light. The green light can indicate the device is working properly and the orange light can indicate that there is some issue with the pump (such as, leak, saturation level of the dressing, blockage downstream of the pump, exhaust blockage, low battery, or the like).

The power source 268 can be in electrical communication with the circuit board 276. One or more power source connections are connected to a surface of the circuit board 276. The circuit board 276 can have other electronics incorporated within. For example, the circuit board 276 may support various sensors including, but not limited to, one or more pressure sensors, temperature sensors, optic sensors and/or cameras, and/or saturation indicators.

FIG. 3 illustrates an electronics assembly 300 enclosing an electronics unit within a housing. As illustrated in FIG. 3, the housing of the electronics assembly 300 can include a plate 301 and flexible film 302 enclosing the electronics unit 303 within. The electronics unit 303 can include a pump 305, inlet protection mechanism 310, pump exhaust mechanism 306, power source 307, and circuit board 309. The circuit board 309 can be flexible or substantially flexible.

As is illustrated, the pump exhaust mechanism 306 can be an enclosure, such as a chamber. The electronics unit 303 and pump 305 can be used without the inlet protection mechanism 310. However, the pump exhaust mechanism 306 and the pump 305 can sit within an extended casing 316.

The flexible film 302 can be attached to the plate 301 to form a fluid tight seal and enclosure around the electronic components. The flexible film 302 can be attached to the plate at a perimeter of the plate by heat welding, adhesive bonding, ultrasonic welding, RF welding, or any other attachment or bonding technique.

The flexible film 302 can include an aperture 311. The aperture 311 can allow the inlet protection mechanism 310 to be in fluid communication with the absorbent and/or transmission layers of the wound dressing. The perimeter of the aperture 311 of the flexible film 303 can be sealed or attached to the inlet protection mechanism 310 to form a fluid tight seal and enclosure around the inlet protection mechanism 310 allowing the electronic components 303 to remain protected from fluid within the dressing. The flexible film 302 can be attached to the inlet protection mechanism 310 at a perimeter of the inlet protection mechanism 310 by heat welding, adhesive bonding, ultrasonic welding, RF welding, or any other attachment or bonding technique. The inlet protection mechanism 310 can prevent wound exudate or liquids from the wound and collected in the absorbent area 160 of the wound dressing from entering the pump and/or electronic components of the electronics assembly 300.

The electronics assembly 300 illustrated in FIG. 3 can be incorporated within the wound dressing such that, once the dressing is applied to the body of the patient, air from within the dressing can pass through the inlet protection mechanism 310 to be pumped out toward the pump exhaust mechanism 306 in communication with an aperture in the casing 316 and the circuit board 309 as described herein.

FIGS. 4A-B illustrate an electronics assembly 400 including a pump inlet protection mechanism 410 sealed to the exterior of the flexible film 402, similar to the description with reference to FIG. 3. Also shown is an exhaust mechanism 406, which can be similar to the exhaust mechanism 306.

FIG. 4A illustrates lower, wound facing surface of the electronics assembly 400. FIG. 4B shows an upper surface of the plate 401 (which can face the patient or user) of the electronics assembly 400. The upper surface of the plate 401 can include an on/off switch or button cover 443 (illustrated as a pull tab), indicators 444, and/or one or more vent holes 442. Removal of the pull tab 443 can cause activation of the electronics assembly 400, such as provision of power from the power source to the electronics assembly. Further details of operation of the pull tab 443 are described in PCT International Application No. PCT/EP2018/079745, filed Oct. 30, 2018, titled “SAFE OPERATION OF INTEGRATED NEGATIVE PRESSURE WOUND TREATMENT APPARATUSES,” which is incorporated by reference in its entirety herein.

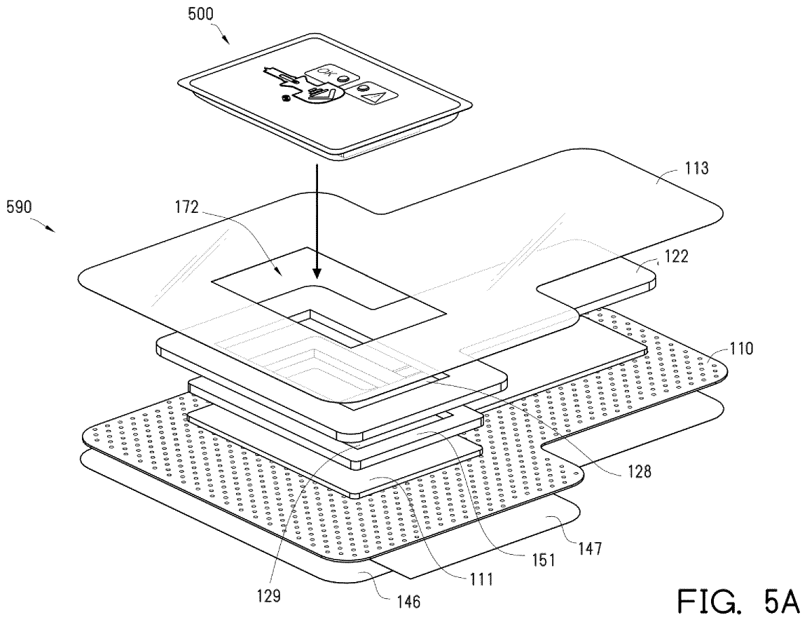

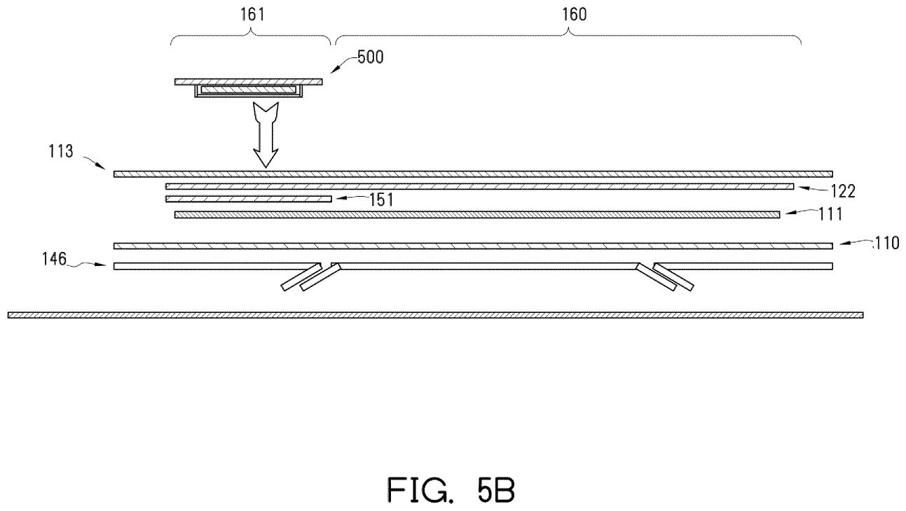

The electronics assembly 400 with the pump inlet protection mechanism 410 extending from and sealed to the film 402 can be positioned within the aperture 172 in the cover layer 113 and absorbent layer(s) (122, 151) as shown in FIG. 1C. The perimeter of the electronics assembly 400 can be sealed to a top surface of the outer perimeter of the aperture 172 in the cover layer 113 as shown in FIGS. 1C and described in more detail with reference to FIG. 5A-5B herein. The electronics assembly 400 can be sealed to the cover layer 113 with a sealant gasket, adhesive, heat welding, adhesive bonding, ultrasonic welding, RF welding, or any other attachment or bonding technique. The electronics assembly 400 can be permanently sealed to the cover layer 113 and could not be removed from the cover layer without destroying the dressing.

The electronics assembly 400 can be utilized in a single dressing and disposed of with the dressing. In some cases, the electronics assembly 400 can be utilized in a series of dressings.

FIG. 5A illustrates a wound dressing, such as the one in FIG. 1C, incorporating an electronics assembly 500 within the wound dressing layers 590. FIG. 5B illustrates a cross-sectional view of the wound dressing incorporating the electronics assembly of FIG. 5A. The electronics assembly 500 can be provided within the aperture 172 in the cover layer and apertures 129 and 128 in the first and second absorbent layers 122, 151. The electronics assembly 500 can seal to the outer perimeter of the aperture 172 of the cover layer. The dressing can comprise a wound contact layer 110 and a moisture vapor permeable film, cover layer or backing layer 113 positioned above the contact layer 110 and other layers of the dressing. A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. Further, one or more absorbent layers (such as layers 122, 151) for absorbing and retaining exudate aspirated from the wound can be utilized. The one or more layers 122, 151 of absorbent material may be provided above the transmission layer 111. There may be a small aperatured absorbent layer 151 and a large aperture absorbent layer 122. The small apertured absorbent layer 151 can be positioned on top of the large apertured absorbent layer 122. In some cases, the small apertured absorbent layer 151 can be positioned below of the large apertured absorbent layer 122. Before use, the dressing can include one or more delivery layers 146 adhered to the bottom surface of the wound contact layer. The delivery layer 146 can cover adhesive or apertures on the bottom surface of the wound contact layer 110.

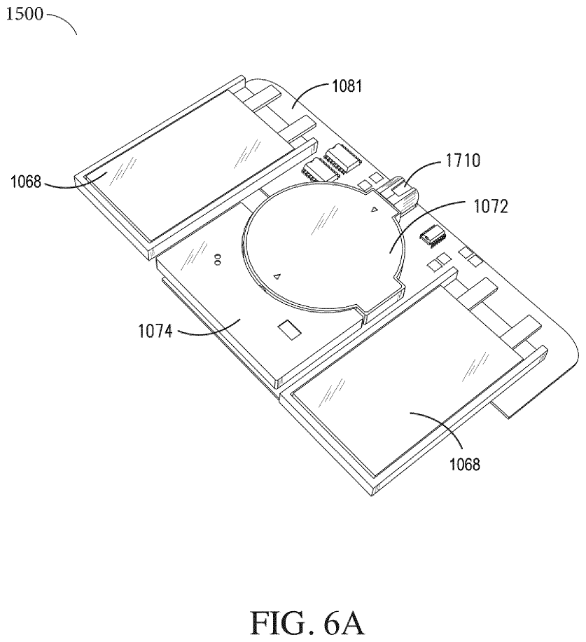

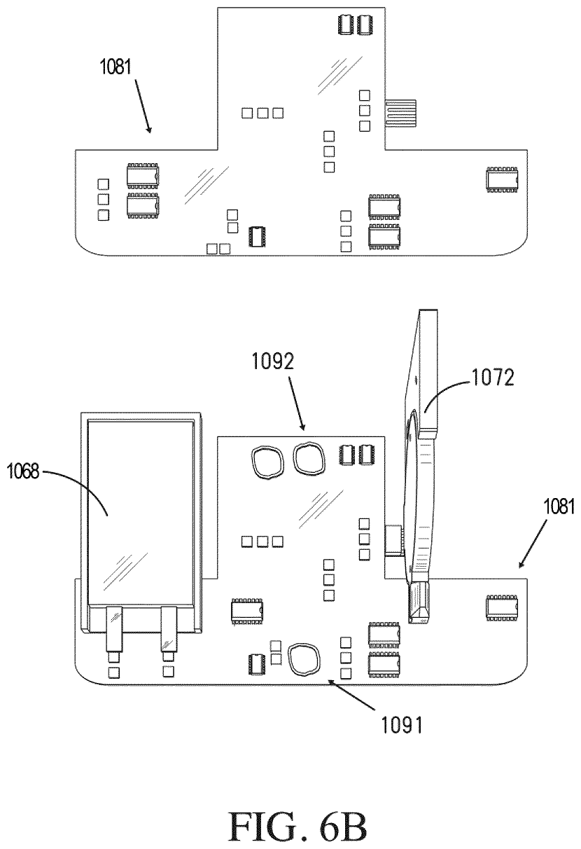

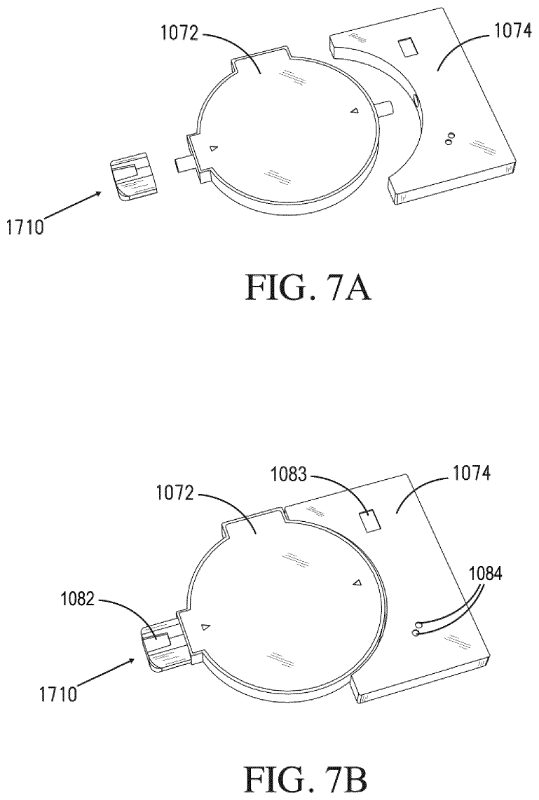

FIGS. 6A-6B and 7A-7B illustrate an electronics assembly 1500 with a pump inlet protection mechanism 1710 and pump exhaust mechanism 1074 on a pump 1072. The assembly 1500 can include cavities 1082 and 1083 (shown in FIGS. 7A-7B) on the pump inlet protection mechanism 1710 and pump exhaust mechanism 1074, respectively. The inlet protection and pump exhaust mechanisms can be adhered to the inlet and the outlet of the pump as described herein. The assembly 1500 can be assembled using an adhesive and allowed to cure prior to incorporating into the electronics assembly.

The pump inlet can be covered or fitted with a pump inlet protection mechanism 1710. The pump inlet protection 1710 can be pushed onto the pump inlet as illustrated by the arrows in FIG. 7A. This can be a friction fit. The port of the pump inlet protection 1710 that receives a portion of the pump inlet can be sized and shaped to be a complementary fit around the pump inlet. The pump inlet protection 1710 can be bonded onto the pump inlet using a silicone sealant or any other sealant or sealing technique. FIG. 7B illustrates the pump inlet protection mechanism 1710 covering the pump inlet and the pump exhaust mechanism 1074 covering the pump outlet. The pump exhaust mechanism 1074 can include one or more apertures or vents 1084 to allow gas aspirated by the pump to be exhausted from the pump exhaust mechanism 1074. In some cases, a non-return valve and/or filter membrane of the pump exhaust mechanism is included in the pump exhaust mechanism 1074.

FIGS. 7A-7B illustrate the pump inlet protection mechanism 1710 and pump exhaust mechanism 1074 with cavities 1082 and 1083. A pump assembly including the pump inlet protection mechanism 1710 and pump exhaust mechanism 1074 can be placed over the surface of a circuit board 1081. When the pump assembly is in contact with the surface of the circuit board 1081, the cavities 1082 and 1083 can at least partially enclose sensors on the circuit board 1081, for example, pressure sensors 1091 and 1092 on the circuit board 1081, as illustrated in FIG. 6B.

The pressure sensors 1091 and 1902 illustrated in FIG. 6B can be used to measure and/or monitor the pressure level at the wound and atmospheric pressure. The pressure sensor 1091 can be used to measure and/or monitor pressure at the wound (such as, underneath the wound dressing), which can be accomplished by measuring and/or monitoring pressure in a fluid flow path connecting the negative pressure source or pump 1072 and the wound. The pressure sensor 1091 can measure and/or monitor pressure in the cavity 1082 of the pump inlet protection mechanism 1710 shown in FIGS. 7A-7B. A power source 1068 (illustrated as two batteries in FIG. 6A) can provide power to the negative pressure source 1072 and the electronics.

The pressure sensor 1092 can be used to measure and/or monitor pressure external to the wound dressing. The pressure sensor 1092 can measure and/or monitor pressure in the cavity 1083 of the pump exhaust mechanism 1074 shown in FIGS. 7A-7B. The pressure sensor 1092 can measure pressure external to the wound dressing, which can be relative atmospheric pressure since the atmospheric pressure varies depending on, for instance, an altitude of use or pressurized environment in which the TNP apparatus may be used. These measurements can be used to establish a desired negative pressure differential (or set point) at the wound relative to the external pressure.

The circuit board 1081 (including any of the circuit boards described herein) can include control circuitry, such as one or more processors or controllers, that can control the supply of negative pressure by the negative pressure source 1072 according at least to a comparison between the pressure monitored by the pressure sensor 1091 and the pressure monitored by the pressure sensor 1092. Control circuitry can operate the negative pressure source 1072 in a first mode (that can be referred to as an initial pump down mode) in which the negative pressure source 1072 is activated to establish the negative pressure set point at the wound. The set point can be set to, for example, a value in the range between about −70 mmHg to about −90 mmHg, among others. Once the set point has been established, which can be verified based on a difference between pressure measured by the pressure sensor 1091 (or wound pressure) and pressure measured by the pressure sensor 1092 (or external pressure), control circuitry can deactivate (or pause) operation of the negative pressure source 1072. Control circuitry can operate the negative pressure source 1072 is a second mode (that can be referred to as maintenance pump down mode) in which the negative pressure source 1072 is periodically activated to re-establish the negative pressure set point when the wound is depressurized as a result of one or more leaks. Control circuitry can activate the negative pressure source 1072 in response to the pressure at the wound (as monitored by the pressure sensor 1091) becomes more positive than a negative pressure threshold, which can be set to the same negative pressure as the set point or lower negative pressure.

Embodiments of the wound dressings, wound treatment apparatuses and methods described herein may also be used in combination or in addition to one or more features described in PCT International Application No. PCT/EP2017/060464, filed May 3, 2017, titled NEGATIVE PRESSURE WOUND THERAPY DEVICE ACTIVATION AND CONTROL, U.S. Pat. Nos. 8,734,425, and 8,905,985, each of which is hereby incorporated by reference in its entirety herein.

One or more self-adhesive gaskets can be applied to the pump inlet protection mechanism 1710 and pump exhaust mechanism 1074 to seal the cavities 1082 and 1083 of the pump inlet and pump exhaust around sensors on the circuit board 1081 and to seal around the exhaust mechanism vent(s) and corresponding vent(s) in the circuit board 1081 (as described herein). A pre-formed adhesive sheet can be used to form the sealing gaskets between the cavities 1082 and 1083 of the pump inlet and pump exhaust mechanisms and sensors on the circuit board 1081 and between the exhaust mechanism vent(s) and vent(s) in the circuit board 1081. In some cases, an adhesive can be used to seal the cavities 1082 and 1083 of the pump inlet protection 1710 and pump exhaust mechanism 1074 around sensors on the circuit board 1081 and to seal around the exhaust mechanism vent(s) 1084 and corresponding vent(s) in the circuit board. As described herein, the electronics assembly 1500 can be embedded within layers of the dressing, such as in cutouts or recesses into which the electronics assembly can be placed.

The pump inlet protection mechanism 1710 can provide a large surface area available for vacuum to be drawn by the inlet of the pump. A pump inlet (shown as rounded protrusion in FIG. 7A) can fit within a recess in the pump inlet protection mechanism 1710. The pump inlet can be attached by friction fit and/or form a complementary fit to the recess of the pump inlet protection mechanism.

The pump inlet protection mechanism 1710 can allow air or gas to pass through, but can block liquid from reaching the negative pressure source. The pump inlet protection mechanism 1710 can include a porous material. The pump inlet protection mechanism 1710 can comprise one or more porous polymer molded components. The pump inlet protection mechanism 1710 can include hydrophobic or substantially hydrophobic material. Material included in the pump inlet protection mechanism 1710 can have a pore size in the range of approximately 5 microns to approximately 40 microns. In some cases, the pore size can be approximately 10 microns. The pump inlet protection mechanism 1710 can include a polymer that can be one of hydrophobic polyethylene or hydrophobic polypropylene. In some cases, the pump inlet protection mechanism can include a Porvair Vyon material with a pore size of 10 microns. Any of the pump inlet protection mechanism described herein can include one or more features of the pump inlet protection mechanism 1710.

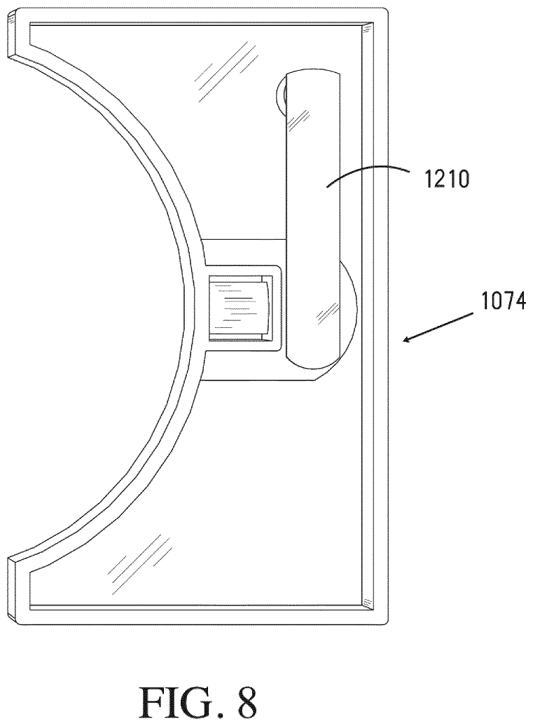

The pump exhaust mechanism 1074 (or any of the pump exhaust or outlet mechanisms described herein) can include a check valve or a non-return valve 1210 as shown in FIG. 8. The non-return valve 1210 can be any suitable mechanical one-way valve, such as, for example, a reed valve, a duckbill valve, a ball valve, a loose leaf valve or an umbrella valve, among others. The non-return valve can be similar to any of the non-return valves described in PCT International Application No. PCT/EP2017/055225, filed Mar. 6, 2017, titled WOUND TREATMENT APPARATUSES AND METHODS WITH NEGATIVE PRESSURE SOURCE INTEGRATED INTO WOUND DRESSING, which is incorporated by reference herein in its entirety. The pump exhaust mechanism 1074 can be bonded to the outlet of the pump using a sealant, for example a silicone sealant. The outlet or exhaust of the pump exhaust mechanism 1074 can include an antimicrobial film and/or other filter membrane that filters gas exhausted outside the NPWT system, such as to the atmosphere. As illustrated, pump exhaust mechanism 1074 can be an enclosure or chamber that is substantially sealed to prevent ingress of gas or fluid other than through the vent(s) 1084.

Any of the embodiments described herein can additionally or alternatively include one or more features described in International Application No. PCT/EP2018/074694, filed Sep. 13, 2018, titled NEGATIVE PRESSURE WOUND TREATMENT APPARATUSES AND METHODS WITH INTEGRATED ELECTRONICS, International Application No. PCT/EP2018/074701, filed Sep. 13, 2018, titled NEGATIVE PRESSURE WOUND TREATMENT APPARATUSES AND METHODS WITH INTEGRATED ELECTRONICS, International Application No. PCT/EP2018/079345, filed Oct. 25, 2018, titled NEGATIVE PRESSURE WOUND TREATMENT APPARATUSES AND METHODS WITH INTEGRATED ELECTRONICS, International Application No. PCT/EP2020/056317, filed Mar. 10, 2020, titled EXHAUST BLOCKAGE DETECTION FOR NEGATIVE PRESSURE WOUND TREATMENT APPARATUSES, each of which is incorporated by reference herein in its entirety.

### Intelligent Electronic Fuse

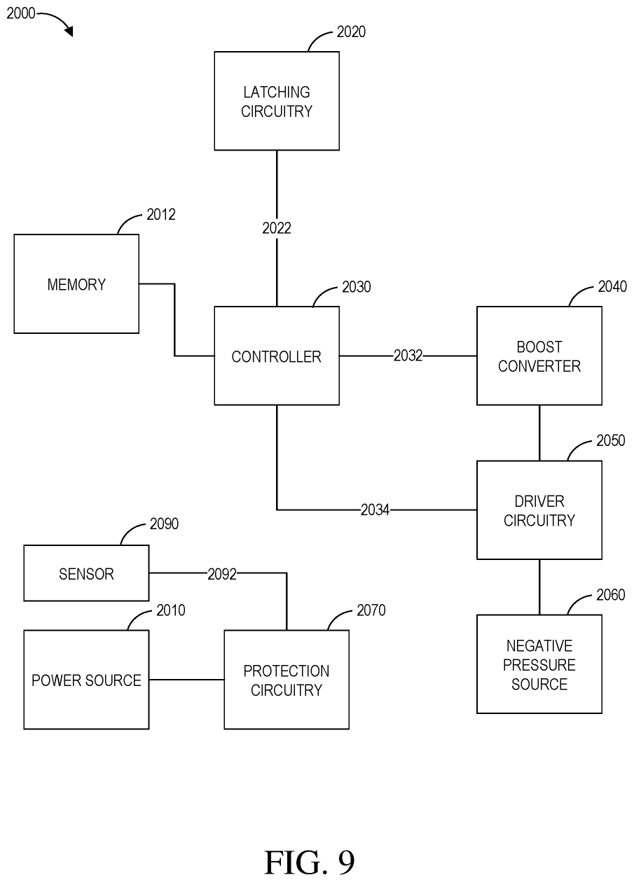

FIG. 9 illustrates a block diagram of the electronic circuitry or electronics 2000 of a electronics components) can include a power source 2010, latching circuitry 2020, a controller 2030, a memory 2012, a boost converter (or regulator) 2040 (sometimes referred to as boost converter circuitry or boost regulator circuitry), driver circuitry 2050, and a negative pressure source 2060. The negative pressure source 2060 can include a piezoelectric pump (such as, a pump operated by a piezoelectric actuator or transducer). In some cases, the driver circuitry 2050 can include H-bridge circuitry. The power source 2010 may include one or more batteries (such as, two 3V batteries). The power source 2010 may provide power to the other electronics components. The power source 2010 (as well as any other electronic component(s)) can be small in case of a wearable TNP system.

Latching circuitry 2020 can include one or more active components (such as, transistors) that activate any one or more of the other components illustrated in FIG. 9 responsive to activation of the TNP device, such as removal of the pull tab. Activation can cause the latching circuitry 2020 to facilitate provision of power from the power source 2010 to one or more of the other components, such as to the controller 2030. In some cases, the latching circuitry 2020, responsive to activation, can provide an indication or signal 2022 to the controller 2030. The latching circuitry 2020 can transition from an inactive state (which can be a default state) to an active state responsive to the activation. Responsive to receiving the signal 2022, the controller 2030 can operate the negative pressure source 2060. For example, the controller can operate the boost converter 2040 via a signal 2032 (such as, a reference voltage or current) and the driver circuitry 2050 via a signal 2034 (such, as a pulse width modulation signal). Further details of operation of the latching circuitry are described in International Publication No. WO 2019/086475, filed Oct. 30, 2018, titled “SAFE OPERATION OF INTEGRATED NEGATIVE PRESSURE WOUND TREATMENT APPARATUSES,” which is incorporated by reference in its entirety herein.

Power supplied by the power source 2010 (such as, 3V or less or more, 4V or less or more, 5V or less or more, 6V or less or more, 7V or less or more, 8V or less or more, 9V or less or more, or the like) may need to be increased for powering the negative pressure source 2060. The boost converter 2040 can increase the power provided by the power source 2010 to a power level adequate for powering the negative pressure source 2060. The boost converter 2040 can include electronic circuitry configured to generate a higher level of power (for example, higher voltage of DC power) from a lower input power (for example, battery power). In some cases, the boost converter 2040 can be a switched-mode power supply. The boost converter 2040 can be a DC to DC converter with an output voltage greater than the input or source voltage. The boost converter 2040 can increase or step up the power level of the power source 2010 to a power level for operating the negative pressure source 2060. For example, the power source 2010 may provide 6V DC power (or less or more) and the negative pressure source 2060 can require DC power between 30V (or less or more) and 22V (or less or more).

The negative pressure source 2060 can be controlled by alternating periods of activation and deactivation of the negative pressure source. A duty cycle of the negative pressure source 2060 can reflect a portion of time during which the negative pressure source is active relative to a given time interval (such as, 1 second, 5 seconds, 10 seconds, 20 seconds, 30 seconds, etc.). For example, if the negative pressure source 2060 is active for 15 seconds over a 30 second time interval, the duty cycle is 50%. In some cases, increase in the duty cycle of the negative pressure source 2060 can be indicative or a presence of one or more leaks (for example, in the seal between the dressing and the patient). This may be due to the negative pressure source 2060 being active longer in order to establish or maintain a negative pressure set point in presence of one or more leaks. Further details of operation of the boost converter and operation of the negative pressure source are described in International Publication No. WO 2020/239781, filed Apr. 26, 2020, titled “SYSTEMS AND METHODS FOR EXTENDING OPERATIONAL TIME OF NEGATIVE PRESSURE WOUND TREATMENT APPARATUSES,” which is incorporated by reference in its entirety herein.

One or more of the electronic components illustrated in FIG. 9 can experience a temperature increase during operation. In some cases, the temperature of the power source 2010 may increase due to a malfunction or misuse of the TNP system. In some instances, the malfunction or misuse of the TNP system can cause a temperature increase of one or more electronic components, such as a boost converter 2040, a driver circuitry 2050, or a negative pressure source 2060. For example, the malfunction or misuse of the TNP system (such as, bending or flexing the TNP system, which can result in a short circuit as described herein) may cause an excessive current or reverse polarity current. As described herein, because the TNP system can be a wearable device, the electronics 2000 can be enclosed in a liquid proof enclosure as well as tightly packaged to reduce the form factor. Such packaging can cause the electronics to generate more heat, which can further exacerbate any temperature increases.

Since the TNP system can be worn by a patient, the temperature increase may cause discomfort or even injury to the patient. As a result, the TNP system temperature may need to be kept below a maximum temperature threshold (such as about 40 degrees Celsius or less or more, 41 degrees Celsius or less or more, 42 degrees Celsius or less or more, 48 degrees Celsius or less or more, 50 degrees Celsius or less or more, 60 degrees Celsius or less or more, 70 degrees Celsius or less or more, 80 degrees Celsius or less or more, 90 degrees Celsius or less or more, or the like). For example, a temperature increase to 41 degrees Celsius can cause a patient to experience a burn and 43 degrees Celsius can severely injure the patient.

To protect the patient against excessive current or reverse polarity current and resulting temperature increase, a conventional fuse can be utilized. However, using a fuse can undesirably increase power consumption (for instance, due to impedance of the fuse) and provide unreliable protection (for example, due to the fuse's fixed current rating or insufficient current provided by a small power source 2010 to blow the fuse).

With reference to FIG. 9, protection circuitry 2070 can protect a patient from injury due to the temperature increase. The protection circuitry 2070 can comply with the IEC 60601-1 standard for medical devices (or similar standard). The protection circuitry 2070 can monitor the temperature and perform one or more remedial actions responsive to detection of the temperature satisfying a threshold indicative of excessive temperature. For instance, a remedial action can include disconnecting the power source 2010 from any of the electronic components (or from a subset of the electronic components or from all of the electronic components). The protection circuit 2070 can monitor current and disconnect the power source 2010 responsive to detection of the current satisfying a threshold indicative or excessive current. The protection circuitry 2070 can be prevent reverse polarity current flow and prevent excessive current flow. Disconnecting the power source 2010 can limit the current provided by the power source 2010. Any of the remedial actions described herein can include providing one or more indications, which can be visual, audible, tactile, etc.

In some cases, the protection circuitry 2070 can monitor temperature of the power source 2010. A sensor 2090 can sense temperature 2092 of the power source 2010. The sensor 2090 can include multiple sensors. The protection circuitry 2070 can receive the monitored temperature 2092. As illustrated in FIG. 9, in some cases, sensor 2090 can be positioned proximal to the power source 2010. In some cases, the sensor 2090 can be part of the protection circuitry 2070. Although FIG. 9 illustrates the protection circuitry 2070 monitoring the temperature of the power source 2010, the protection circuitry 2070 can alternatively or additionally monitor the current provided by the power source 2010 or temperature (or current) of another electronic component, such as the boost converter 2040, driver circuitry 2050, or negative pressure source 2060). For example, to monitor current provided by the power source 2010 the sensor 2090 can additionally or alternatively include a resistor.

The protection circuitry 2070 can be provided in a form of an integrated circuit (IC). This can facilitate testing and certification of performance of the protection circuitry 2070, which in turn can facilitate testing and certification of the TNP device (for example, as complying with IEC 6060-1 or similar standard). The protection circuitry 2070 can dynamically adjust to the threshold temperature and current of the particular TNP system. The protection circuitry 2070 can be programmable (for instance, can include one or more controllers). The protection circuitry 2070 can be resettable following detection and correction of an overtemperature or overcurrent event so that the TNP device can continue to operate to provide therapy to the patient. In some cases, the protection circuitry 2070 can be an electronic fuse (eFuse).

Advantageously, the protection circuitry 2070 can monitor temperature of one or more electronic components external to the protection circuitry and perform one or more remedial actions responsive to one or more of increasing temperature or current. The protection circuitry 2070 can configured (such as, programmed) to respond to one or more particular thresholds. For protection against excessive current, the protection circuitry 2070 can be configured to respond to small currents consumed by a wearable TNP system, such as between about 150 mA and 300 mA.

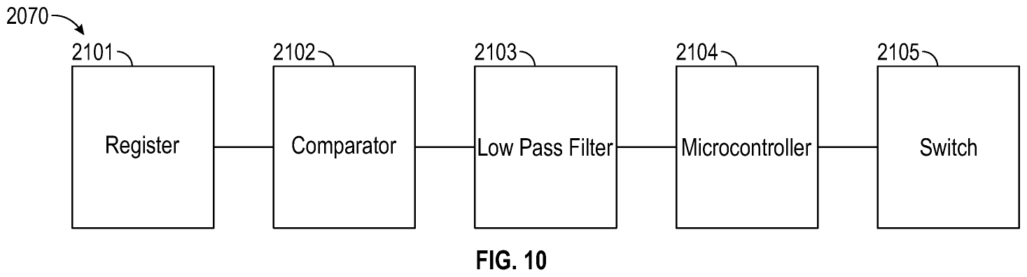

FIG. 10 illustrates a block diagram of a protection circuitry, such as the protection circuitry 2070. The protection circuitry 2070 can include a register 2101, a comparator 2102, a low pass filter 2103, a controller 2104 (which can be a microcontroller), and a switch 2105. In some cases, one or more of the register 2101, the comparator 2102, or the low pass filter 2103 can be integrated into the microcontroller 2104. The register 2101 can be connected to the sensor 2090 configured to sense temperature of the power source 2010. The comparator 2102 can be configured to compare the value(s) stored in the register 2101 to a threshold (such as, 41 degrees Celsius). The low pass filter 2103 can be configured to remove noise from the output of the comparator 2102. The microcontroller 2104 can respond to the output of the comparator 2102 (which can be filtered by the low pass filter 2103). For instance, responsive to the output of the comparator 2102 indicating that the temperature of the power source 2010 satisfies the temperature threshold, the microcontroller 2104 can open the switch 2105 to disconnect the power source 2010 from the other electronic components. To conserve power, the microcontroller 2104 can by default operate in low power mode (such as, in sleep mode). Responsive to the output of the comparator 2102 indicating that the temperature of the power source 2010 satisfies the temperature threshold, the microcontroller 2104 can transition to a higher power mode (such as, wake up). This operation can be accomplished by triggering an interrupt of the microcontroller 2104, causing the microcontroller 2104 to wake up. In some cases, multiple comparators can be present to facilitate detection for multiple temperature thresholds.

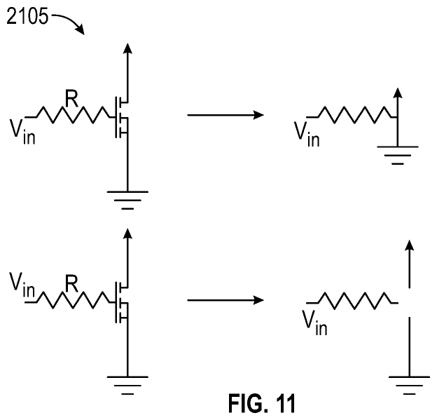

With reference to FIG. 11, the switch 2105 can be a transistor (such as, a field effect transistor (FET)). When an input voltage Vin is higher than the threshold voltage of the FET), the switch 2105 can be closed (as illustrated in the top portion of FIG. 11) and current can flow from the power source 2010 to the other electronic components. When the microcontroller 2104 detects an excessive temperature, the microcontroller 2104 can open the switch 2105 by limiting the input voltage to a voltage lower than the threshold voltage of the FET (as illustrated in the bottom portion of FIG. 11). This can prevent the flow of current from the power source 2010. Advantageously, a FET switch 2105 can have smaller impedance (and consume less current) than a fuse, as explained below in connection with the switch 1520. Using a low impedance switch 2105 can be particularly advantageous for a wearable TNP system that operates with small current (such as, between about 150 mA and 300 mA as described herein).

In some implementations, responsive to overtemperature detection a switch configured to control negative pressure wound therapy (such as, the switch 265 as illustrated in FIG. 2A) can be deactivated. Deactivation of such switch can be performed by the microcontroller 2104.

As described herein, the microcontroller 2104 can perform one or more remedial actions based on comparing sensed temperature to one or more temperature thresholds. For example, the microcontroller 2104 can cause a deactivation of the negative pressure source 2060 (such as, disconnecting the power source 2010 by opening the switch 2105) responsive to a determination that the sensed temperature satisfies the temperature threshold. Including an independent and redundant mechanism for responding to temperature increases can be advantageous for ensuring the safe and effective provision of negative pressure wound therapy to the patient.

Advantageously, the protection circuitry 2070 can facilitate distinguishing between different operating conditions and performing different remedial actions. For example, assume that the sensor 2090 (as shown in FIG. 9) detects temperature that satisfies a temperature threshold that is lower than the threshold indicative of temperature dangerous for the patient (such as, 37 degrees Celsius) and that the current (such as, the current provided by the power source 2010) is within normal operating range. Such operating condition is likely caused by the TNP system being proximal to something hot (such as, a hot object). The microcontroller 2104 can provide a first indication to the controller 2030 to take a remedial action, such as slowing down therapy (to lower the temperature) or stopping therapy. As another example, assume that the temperature sensed by the sensor 2090 is within the normal operating range and that the current is increasing (for instance, satisfies a current threshold). Such operating condition is likely caused by the TNP system not performing correctly. The microcontroller 2104 can provide a second indication to the controller 2030 to take a remedial action, such as stopping therapy and performing one or more self-checks or tests. As yet another example, assume that the temperature sensed by the sensor 2090 satisfies the temperature threshold (or a higher temperature threshold, such as 41 degrees Celsius) and the current satisfies the current threshold. Such operating condition is likely caused by a short circuit, liquid ingress (such as, one or more of exudate or blood), or another malfunction. The microcontroller 2104 can open the switch 2105 to protect the patient.

As described herein, for a wearable TNP system, current consumption can be small. In some cases, the current can be between about 150 mA and 300 mA. The current threshold can be about 320 mA.

In some cases, current consumption of the TNP system can be characterized, for instance, in manufacturing. For instance, current consumption can be separately characterized in the initial pump down and maintenance pump down modes. Maximum current consumption for each of the pump down modes can be recorded (and stored in memory). Subsequently, during provision of negative pressure therapy to a patient, current consumption can be monitored by the protection circuitry 2070, as described herein. One or more remedial actions can be taken (such as, stopping therapy) responsive to current satisfying one or more maximum current consumption thresholds previously determined during characterization. Higher current consumption can be indicative of liquid ingress.

Due to the flexible, configurable nature of the protection circuitry 2070, any of the temperature or current thresholds can be configurable. For instance, in some cases, the microcontroller 2104 can be configured (such as, programmed) to permit the TNP system to provide therapy at higher (or lower) temperatures or currents.

In some cases, the electronics 2000 can include a separate power source for the protection circuity, such as a supercapacitor. Even if the power source 2010 is disconnected as described herein, the microcontroller 2104 can be powered by the separate power source. The protection circuitry 2070 can include memory (or utilize memory 2012) and the memory 2012 so that the microcontroller 2104 can store indications and other debugging data even when the controller 2030 does not receive power. Such stored data can be utilized to determine the cause of malfunction.

Further details of temperature monitoring and control are described in the International Application No. PCT/EP2021/076022, filed on Sep. 22, 2021, titled “TEMPERATURE MONITORING AND CONTROL FOR NEGATIVE PRESSURE WOUND THERAPY SYSTEMS,” which is incorporated by reference in its entirety.

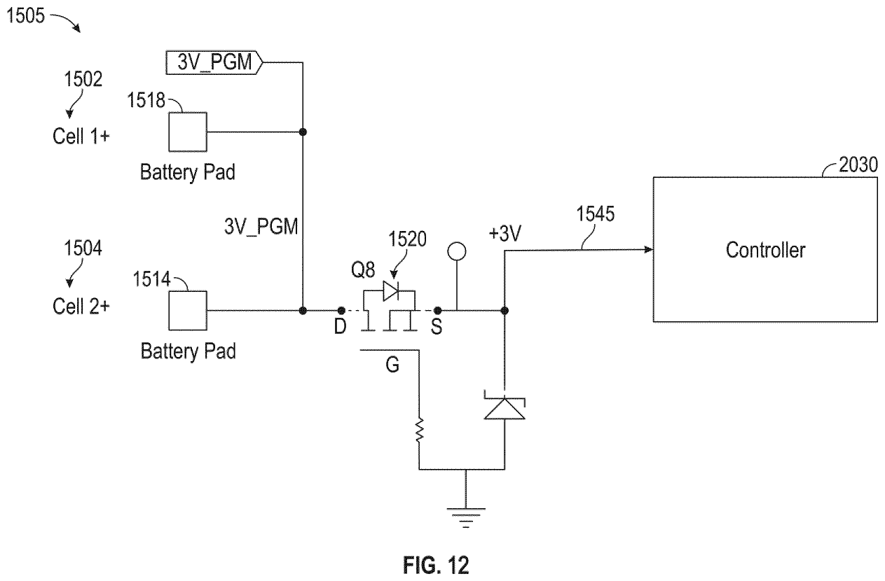

As described herein, the protection circuitry 2070 can provide reverse polarity protection. Reverse polarity can cause a reverse current flow within the electronics 2000 of a TNP system. FIG. 12 that illustrates a circuit 1505 that can include two battery cells 1502 and 1504 (which can correspond to the power source 2010). In some cases, the battery cells 1502 and 1504 can be connected in series to increase the output voltage. For example, each of the battery cells 1502 and 1504 can be a 3V cell, and the combined output of both cells can be 6V. In some cases, the output of one of the battery cells can provide power (labeled as 3V_PGM indicative of 3V low voltage) to one or more electronic components, such as a controller 2030 via the connection 1545 as illustrated in FIG. 12. Low voltage power connection 1545 can be at the same electrical potential as the 3V_PGM terminal described herein. Output of both of the battery cells (or high voltage), which may be enabled by a latching circuit as described herein, can power one or more other electronic components, such as the negative pressure source.

The protection circuitry 2070 can protect the circuit 1505 from excessive current being provided by one or more of the battery cells. As illustrated in FIG. 9, the protection circuitry 2070 can be connected to the terminals of the respective battery cells 1502 and 1504 prior to any other electronic components being connected to the battery cells.

Battery cell 1502 can have positive and negative terminals (only the positive terminal 1518 is illustrated). Battery cell 1504 can have positive and negative terminals (only the positive terminal 1514 is illustrated). Reverse current may undesirably flow from a positive terminal of one of the battery cells into another terminal of the other battery cell (such as, for example from the terminal 1514 into the terminal 1518 or vice versa). Reverse current can be cause by flexing of the circuit board, which can cause shorting the circuit ground connection to the high voltage supply rail (which can be connected to the positive terminal 1518). As described herein, flow of reverse current may be undesirable since it can increase temperature or damage of one or both of the battery cells or any of the other circuit components. For example, flow or reverse current can cause discomfort to the patient, burn the patient, or otherwise compromise patient comfort or safety as well as start a fire.

With reference to FIG. 12, a switch 1520 can provide reverse current (or reverse polarity) protection in the illustrated circuit 1505. As shown, the switch 1520 can be a transistor (such as, p-channel FET or a PNP transistor) with a body diode connected in parallel across the transistor. The switch 1520 can be integrated in the protection circuitry 2070. For instance, the switch 2105 can function as the switch 1520. Under a normal operating condition, the body diode would be forward-biased (or conducting), which would place the source terminal(S) of the switch 1520 at about 2.4V and the gate terminal (G) at about 0V. As a result, the switch 1520 would be turned on, thereby allowing the current to flow across the transistor (whose internal resistance can be relatively small, such as about 50 mOhm or less or more) and bypass the body diode. Under a fault condition (such as, when the circuit ground connection is shorted to the high voltage supply rail, as described herein), the gate terminal (G) voltage would be greater than or equal to the source terminal(S) voltage. As a result, the switch 1520 would be turned off, which can block the flow or the reverse current. In some cases, the body diode can block the flow of the reverse current (for example, since the body diode would be reverse-biased). Accordingly, the switch 1520 and the body diode can provide reverse polarity protection without incurring a diode forward voltage drop. Thereby, full power can be supplied to the one or more electronic components, such as the controller 2030.

In some instances, a diode (such as, a Schottky diode) or a transistor with a resistor can be used as the switch to provide reverse polarity protection (for instance, instead of the transistor and body diode illustrated in FIG. 12). A Schottky diode may have a low forward voltage drop. As a result, when such Schottky diode is forward-biased, less energy would be wasted as heat and efficient power supply to one or more electronic components can be achieved.

Further details of reverse polarity protection are described in International Patent Application No. PCT/EP2022/066993, filed on Jun. 22, 2022, titled “LIQUID INGRESS PROTECTION AND DESIGN OF ELECTRONIC CIRCUITRY FOR NEGATIVE PRESSURE WOUND THERAPY SYSTEMS,” which is incorporated by reference in its entirety.

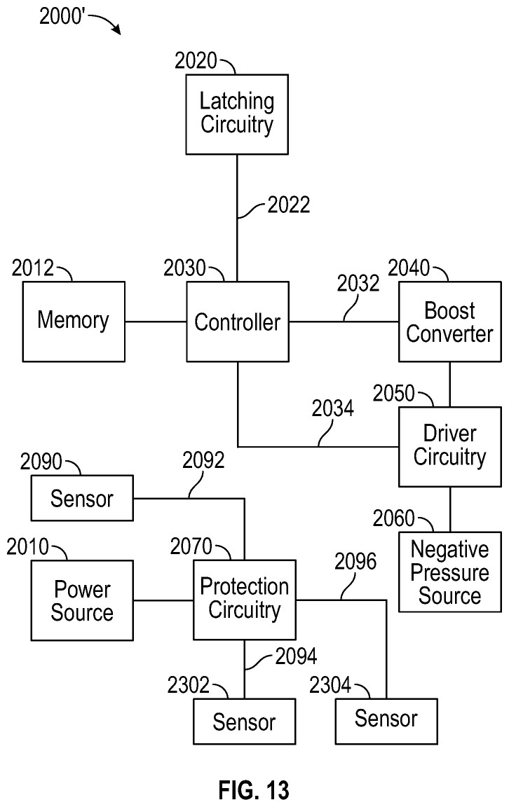

In some cases, as shown in FIG. 13, the protection circuitry 2070 can monitor one or more of temperature or current of various electronic components and take one or more remedial actions. In the electronic circuitry 2000′ illustrated in FIG. 13, which can be similar to the electronic circuitry 2000 (with the exception of the additional sensors 2302 and 2304), there can be three sensors: the sensor 2090, a sensor 2302 configured to monitor temperature of the protection circuitry 2070, and a sensor 2304 configured to monitor temperature of external environment (which can be similar to the sensor 1092, which can monitor both pressure and temperature). The protection circuitry 2070 can utilize the temperature monitored by the sensor 2304 and associated with the external environment for calibration. For example, when the external temperature is high, a temperature threshold for limiting the current may be lowered as compared to a situation when the external temperature is low. Calibration may be particularly important due to the electronic circuitry 2000′ being positioned in a sealed enclosure, which can cause the temperature to rise quickly.

The protection circuitry 2070 can monitor changes of the temperatures obtained by the sensors 2090 and 2302 and perform one or more remedial measures. As described herein, the microcontroller 2104 of the protection circuitry 2070 can detect an increase of the temperature monitored by the sensor 2090 above a first threshold (such as, 41 degrees Celsius) and limit the current provided by the power source 2010 by opening the switch 2105. The protection circuitry 2070 can open the switch 2105 responsive to the detection of an increase of the temperature monitored by the sensor 2302 above a second threshold different than the first threshold (for instance, the second threshold can be greater than the first threshold). Monitoring the temperature increase of the temperature monitored by the sensor 2302 and taking one or more remedial actions can ensure correct and continuous operation of the protection circuitry 2070.

### Endothermic Protection

In addition to or alternatively to the electrical-based approaches for thermal protection described above, one or more chemical-based approaches can be used. Any of the TNP systems described herein can incorporate a thermally triggered endothermic process (or reaction) into the enclosure (which can be a housing) of the TNP system. An endothermic reaction can absorb heat. Responsive to a power source or another electronic component (or components) overheating, the endothermic reaction would be triggered and would suppress the temperature spike, thereby limiting the heat and protecting the patient. For instance, chemical-based approach(es) can serve as a primary mode of thermal protection and electrical-based approach(es) can serve a secondary mode of thermal protection. In such arrangements, therapy can be allowed to continue in cases of smaller temperature increases when electrical-based approach(es) is actively monitoring any temperature increase, while reaching excessive temperature would result in triggering chemical-based approach(es) and causing a TNP system to become non-operational.

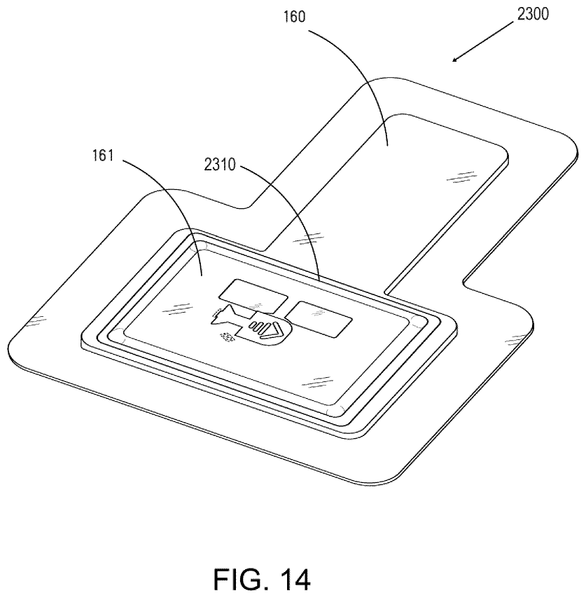

FIG. 14 illustrates a wound dressing 2300 incorporating a TNP system. FIG. 14 is similar to the wound dressing 100 illustrated in FIGS. 1A-1C with the exception of a pouch 2310 that stores the reactants of an endothermic reaction. The pouch 2310 can at least partially surround the electronics positioned in the electronics area 161. The pouch can be positioned between the electronics and the patient (for instance, the pouch can be positioned on a TNP system surface (or side) facing the patient). In some cases, the pouch can be positioned between the electronics and a TNP system surface facing away from the patient particularly in cases the TNP system may experience higher temperatures on the surface facing away from the patient (for instance, due to the positioning of electronic components on that surface, as shown in FIG. 14). Accordingly, the endothermic reaction would be directly triggered by the source of heat as well as mitigate the source of heat rather than merely provide a protective barrier between the source of heat and the patient. In some implementations, the endothermic reaction can be initiated on one side of the TNP system and allow the cooling effect to protect the other side. This can be achieved by including one or more channels or passages through or around a printed circuit board supporting the electronic components or other components (such as, an enclosure protecting the electronics). The printed circuit board can include multiple boards or be folded, labyrinthine, non-planar. In some cases, the enclosure can be folded, labyrinthine, or non-planar. The channels can be formed prior to the initiation of the endothermic reaction or, in some cases, following the initiation of the reaction.

The reactants can include urea salt and water. In some cases, propylene glycol (or another alcohol) and water can be used as reactants. In some instances, sodium chloride (or another salt) and water can be used as reactants.

One or more reactants can be obtained from the chemical components present during negative pressure wound therapy. For example, wound fluid or urine can serve as a reactant. As a result, in case of a breach of the enclosure, which can cause an over-temperature event due to an electrical short circuit, the fluid generating the hazard can become a part of the mitigation against that hazard.

The reactants for the endothermic reaction can be incorporated as part of the structure of the TNP system. For instance, a suitable salt can be absorbed into foamed materials, fluid, or gel (for instance, of a dressing). In some cases, one or more of foam, fluid, or gel may already be present in the dressing to facilitate at least one of absorbency, pressure, pressure relief, or the like. In some implementations, one or more of foam, fluid, or gel may be added to the dressing as part of endothermic temperature relief, and may have one or more secondary uses.

A mechanism for triggering the endothermic reaction (sometimes referred to as a trigger) can be a material that changes shape or moves responsive to a temperature increase. For instance, a bimetal (or bimetallic) switch (such as, a bimetal disk) can be used. The trigger can be configured to change shape or move responsive to a desired temperature threshold indicative of excessive temperature (such as, 41 degrees Celsius). Such transition can result in breaking a separation between the reactants of an endothermic reaction and triggering the reaction. The trigger can be made out of material that returns from a deformed shape to the original shape (such as, a shape-memory alloy, shape-memory polymer, or the like) responsive to a temperature decrease. In some cases, the trigger can be made out of material that undergoes elastomeric expansion responsive to a temperature increase. For example, a temperature junction heating triggering electroactive polymer, shape-memory polymer or alloy, or the like can be used.

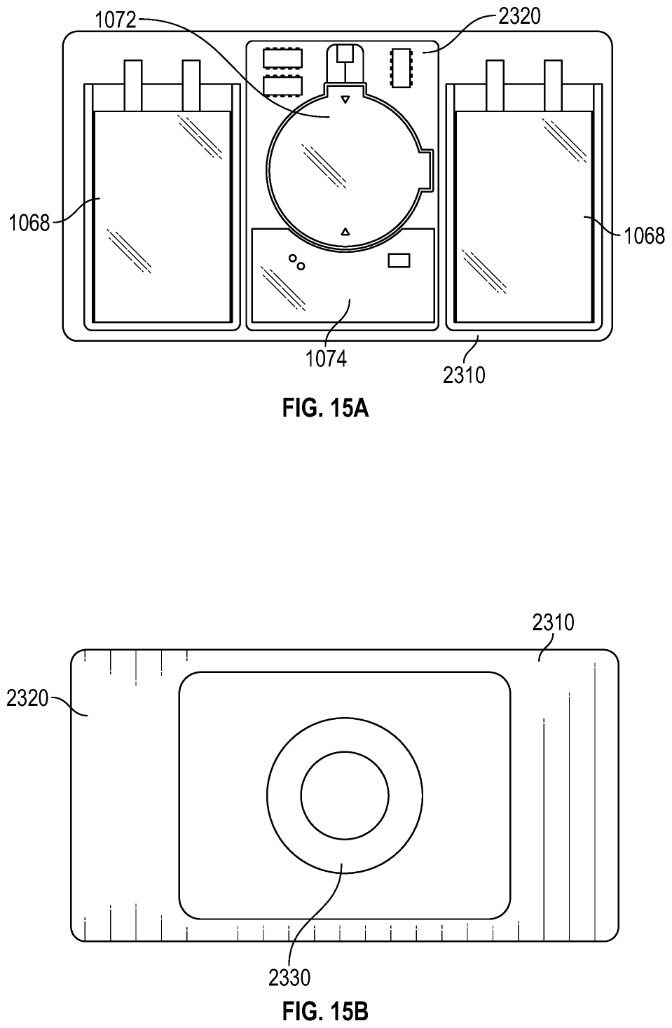

FIGS. 15A and 15B illustrate top and bottom sides of the electronics area 161 and the pouch 2310 of FIG. 14. As described herein, the electronics area 161 can include one or more batteries 1068, pump 1072, and pump exhaust mechanism 1074. A thermal conductor 2320 made out of thermally conductive material (for example, metal, ceramic, aluminum nitride, silicon carbide, etc.) can be provided for conducting heat from one or more batteries, pump 1072, or another electronic component(s) to a trigger, which can be a bimetal disk 2330. The trigger 2330 can be in contact with the thermal conductor 2320 (for instance, be surrounded by the thermal conductor as illustrated in FIG. 15B). The trigger 2330 can be positioned on the bottom side of the electronic area 161 (or in some cases on the top side of the electronics area 161). In some implementations, the thermal conductor 2320 can be omitted. For instance, separate triggers can be positioned under (or otherwise proximal to) each of the batteries 1068 (or one or more electronic components for which thermal protection is being provided). Each trigger can independently activate the endothermic reaction.

In some cases, separate pouches can be positioned under (or otherwise proximal to) each of the batteries 1068 (or one or more electronic components for which thermal protection is being provided). Separate triggers 2330 can be utilized to independently initiate the endothermic reaction in each of the pouches.

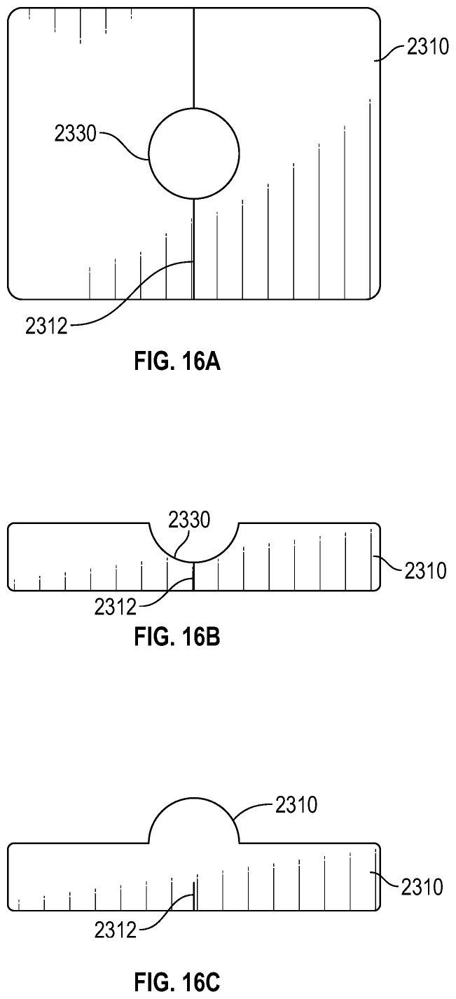

As illustrated in FIGS. 16B-16C, the trigger 2330 (for example, a bimetal disk) can change shape (or snap) from a concave shape to a convex shape (or vice versa) responsive to a temperature increase. The pouch 2310 can include two (or more) sections separated by a partition (or separation) 2312, as illustrated in FIG. 16A. Each of the sections can store one of the reactants for the endothermic reaction, and the partition 2312 can separate the reactants. During normal operation when the temperature is below a temperature threshold, the trigger 2330 can be in a first (or closed) configuration illustrated in FIG. 16B. Responsive to a temperature increase that satisfies the temperature threshold, the trigger 2330 can transition to a second (or open) configuration illustrated in FIG. 16C. This would cause a breach in the partition 2312 and the trigger 2330 and allow the reactants from the two sections to mix, thereby initiating the endothermic reaction. The trigger 2330 can include one or more spikes, edges, or other sharp features to cause the breach (for instance, to pierce or otherwise break the partition 2312).

In some cases, the trigger 2330 can act as a partition. With reference to FIG. 16B, the trigger 2330 can extend across the entire cross-section of the pouch 2310. In such configurations, the partition 2312 may be omitted.

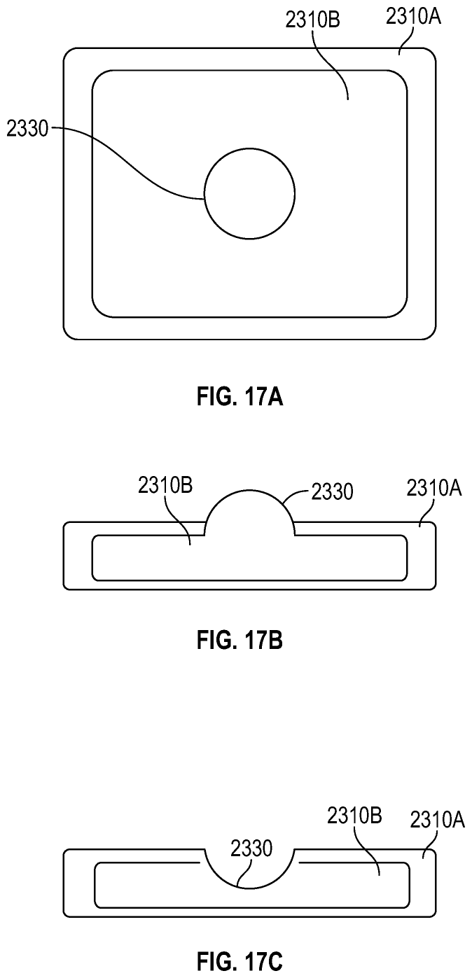

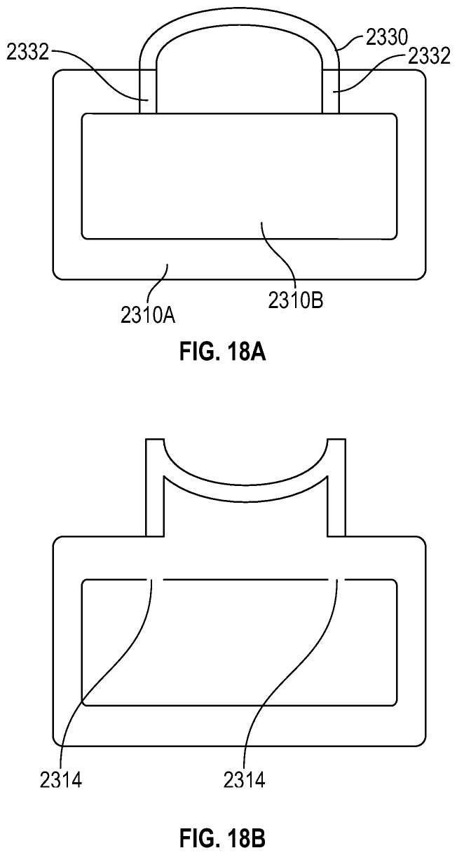

In some implementations, the trigger may be irreversible such that once the trigger has been activated, the TNP system would no longer be usable. For instance, breaching the partition 2312 is an example of an irreversible trigger. In some cases, the trigger can be reversible (see, for instance, FIGS. 17A-17C and FIGS. 18A-18B). Such reversible trigger can re-plug or reconstruct the breach if cooling successfully occurs. Reversible trigger can allow for mitigation of cases when the endothermic reaction generates harmfully low temperatures. Additionally or alternatively, reversible trigger can allow the remaining reactants to be used again in case of one or more subsequent temperature increases.

In some instances, the partition 2312 separating the reactants can be broken directly responsive to a temperature increase. For instance, the separation can be made out of material that softens or melts (such as, a thermoplastic polymer, which can be ABS). The partition 2312 can be made out of material that changes shape (such as, shortens). For instance, a suitable polymer can be used. In such cases, the trigger 2330 may be omitted.

A catalyst for the endothermic reaction can be used. For example, the catalyst can be aluminium oxide or titanium dioxide. The reactants may already be mixed in the pouch 2310 and the pouch 2310 may not need to have two sections to separately store the reactants. The catalyst can be separated from the already mixed reactants, and mixing of the catalyst and the reactants can be triggered using any of the approaches described herein. For instance, the partition 2312 can contain the catalyst and breach of the partition caused by the trigger would release the catalyst. In some cases, the partition 2312 itself can be the catalyst for the endothermic reaction.

With reference to FIGS. 17A-17C, a pouch can include two (or more) nested pouches 2310A and 2310B storing the reactants. When the trigger 2330 snaps to another shape, such as shown in FIG. 17C, the reactants can mix, thereby triggering the endothermic reaction. Alternatively to the trigger 2330, initiating the reaction can be accomplished using any other approach described herein.

In some cases, the trigger 2330 can include one or more tabs (or protrusions) that plug one or more holes in a pouch. Upon changing shape, the one or more tabs can move allowing the reactants to mix through the one or more holes. FIGS. 18A illustrate nested pouches 2310A and 2310B and the trigger 2330 having two tabs 2332. When the trigger 2330 snaps to another shape, such as shown in FIG. 18B, two holes 2314 are exposed allowing the reactants stored in the pouches 2310A and 2310B to mix.

In some cases, different pouches or sections storing the reactants can be broken to allow the materials to mix. In some instances, the temperature increase can directly trigger the endothermic reaction by providing the required activation energy.

The trigger (for example, a bimetal disk) can mechanically alter the structure of the TNP system to move one or more hot components away from being in contact with the patient. This can provide for a greater conduction pathway distance to dissipate the heat and could turn a highly localized temperature (such as, from a hot electronic component) into a lower temperature over a larger area that will not cause harm to the patient. In some cases, the device could dislodge itself from the patient (such as, fall or pop off). This can be utilized as a reversible action independent of the triggering of the endothermic reaction or in addition to the triggering of the reaction. In some cases, the reaction could be used to change the topology of the device to move the one or more hot components away from the patient. The change of topology could be used to indicate that the device has failed or be used as a mechanism to ablate the device off the skin or wound and thus remove the source of hazard from the location in which it can cause harm.

The trigger can be disabled until the first use of the TNP system on the patient (for example, activation as described herein). This can permit the TNP system to undergo one or more elevated temperature processes, such as sterilization. As an example of disabling the trigger, a button or a pull tab (as described herein) can be positioned to limit the motion or position of the trigger.

Additionally or alternatively to the approaches described in this section, a printed circuit board supporting the electronic components (or another adjacent element) can be covered in a material that absorbs the generated heat energy and utilize it to change state rather than elevating temperature. For instance, wax can be used.

Although some of the example in this section refer to the use of a bimetal disc as the trigger other trigger mechanisms can be used.

Advantageously, the approaches described in this section can provide automatic and preemptive thermal protection. The approaches described in this section can provide thermal protection without relying on electronic approaches (such as, one or more fuses). The approaches described in this section can mitigate the effects of a rising temperature anywhere in the TNP system and irrespective of whether the temperature increase has been caused by an electrical malfunction. For example, the approaches described in this section can protect from the potentially harmful effects of radiation from direct sunlight being absorbed by one or more components of the TNP system and retransmitting the energy to the wound or skin of the patient.

### Other Variations

While certain embodiments described herein relate to integrated negative pressure wound therapy systems in which the negative pressure source is supported by the dressing, systems and methods described herein are applicable to any negative pressure wound therapy system or medical system, particularly to systems being positioned on (or worn by) the patient. For example, systems and methods for controlling operation described herein can be used in fluid-proof (such as, water-proof) negative pressure wound therapy systems or medical systems. Such systems can be configured with the negative pressure source and/or electronics being external to the wound dressing, such as with the negative pressure source and/or electronics being positioned in a fluid proof enclosure. Additionally, such systems can be configured to be used within ultrasound delivery devices, negative pressure devices powered by an external power supply, negative pressure devices with a separate pump, and medical devices generally. The approaches described herein are not limited to medical systems and can be used in any wearable electronic system (such as, monitoring and/or therapy system).

Any of the embodiments disclosed herein can be used with one or more features disclosed in U.S. Pat. No. 7,779,625, titled “DEVICE AND METHOD FOR WOUND THERAPY,” issued Aug. 24, 2010; U.S. Pat. No. 7,964,766, titled “WOUND CLEANSING APPARATUS IN SITU,” issued on Jun. 21, 2011; U.S. Pat. No. 8,235,955, titled “WOUND TREATMENT APPARATUS AND METHOD,” issued on Aug. 7, 2012; U.S. Pat. No. 7,753,894, titled “WOUND CLEANSING APPARATUS WITH STRESS,” issued Jul. 13, 2010; U.S. Pat. No. 8,764,732, titled “WOUND DRESSING,” issued Jul. 1, 2014; U.S. Pat. No. 8,808,274, titled “WOUND DRESSING,” issued Aug. 19, 2014; U.S. Pat. No. 9,061,095, titled “WOUND DRESSING AND METHOD OF USE,” issued Jun. 23, 2015; U.S. Pat. No. 10,076,449, issued Sep. 18, 2018, titled “WOUND DRESSING AND METHOD OF TREATMENT”; U.S. patent application Ser. No. 14/418,908,filed Jan. 30, 2015, published as U.S. Publication No. 2015/0190286, published Jul. 9, 2015, titled “WOUND DRESSING AND METHOD OF TREATMENT”; U.S. Pat. No. 10,231,878, titled “TISSUE HEALING,” issued Mar. 19, 2019; PCT International Application PCT/GB2012/000587, titled “WOUND DRESSING AND METHOD OF TREATMENT” and filed on Jul. 12, 2012; International Application No. PCT/IB2013/001469, filed May 22, 2013, titled “APPARATUSES AND METHODS FOR NEGATIVE PRESSURE WOUND THERAPY”; PCT International Application No. PCT/IB2013/002102, filed Jul. 31, 2013, titled “WOUND DRESSING AND METHOD OF TREATMENT”; PCT International Application No. PCT/IB2013/002060, filed Jul. 31, 2013, titled “WOUND DRESSING AND METHOD OF TREATMENT”; PCT International Application No. PCT/IB2013/00084, filed Mar. 12, 2013, titled “REDUCED PRESSURE APPARATUS AND METHODS”; International Application No. PCT/EP2016/059329, filed Apr. 26, 2016, titled “REDUCED PRESSURE APPARATUSES”; PCT International Application No. PCT/EP2017/059883, filed Apr. 26, 2017, titled “WOUND DRESSINGS AND METHODS OF USE WITH INTEGRATED NEGATIVE PRESSURE SOURCE HAVING A FLUID INGRESS INHIBITION COMPONENT”; PCT International Application No. PCT/EP2017/055225, filed Mar. 6, 2017, titled “WOUND TREATMENT APPARATUSES AND METHODS WITH NEGATIVE PRESSURE SOURCE INTEGRATED INTO WOUND DRESSING”; PCT International Application No. PCT/EP2018/074694, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/074701, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/079345, filed Oct. 25, 2018, titled “NEGATIVE PRESSURE WOUND TREATMENT APPARATUSES AND METHODS WITH INTEGRATED ELECTRONICS”; PCT International Application No. PCT/EP2018/079745, filed Oct. 30, 2018, titled “SAFE OPERATION OF INTEGRATED NEGATIVE PRESSURE WOUND TREATMENT APPARATUSES”; each of which is incorporated by reference herein in its entirety.

Although certain embodiments described herein relate to wound dressings, systems and methods disclosed herein are not limited to wound dressings or medical applications. Systems and methods disclosed herein are generally applicable to electronic devices in general, such as electronic devices that can be worn by or applied to a user.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure.

The various components illustrated in the figures or described herein may be implemented as software or firmware on a processor, controller, ASIC, FPGA, or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICS, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A negative pressure wound therapy system comprising:
a wound dressing configured to be placed over a wound, the wound dressing configured to absorb fluid aspirated from the wound;
a negative pressure source disposed on or within the wound dressing, the negative pressure source configured to aspirate fluid from the wound;
an electronic control circuitry disposed on or within the wound dressing and configured to operate the negative pressure source;
a power source disposed on or within the wound dressing and configured to provide power to the negative pressure source and the electronic control circuitry; and
an integrated circuit disposed on or within the wound dressing and configured to receive power from the power source, the integrated circuit comprising an electronic protection circuitry configured to:
monitor a temperature of the power source;
responsive to the temperature satisfying at least one temperature threshold, cause the electronic control circuitry to adjust operation of the negative pressure source or prevent supply of power from the power source; and
responsive detection of at least one of a reverse current condition or an excessive current condition, prevent supply of power from the power source.

2. The negative pressure wound therapy system of claim 1, wherein the electronic protection circuitry comprises a switch configured to be opened to prevent supply of power from the power source.

3. The negative pressure wound therapy system of claim 1, wherein integrated circuit is disposed between the power source and the negative pressure source.

4. The negative pressure wound therapy system of claim 1, wherein the integrated circuit comprises a temperature sensor configured to measure the temperature of the power source.

5. The negative pressure wound therapy system of claim 1, further comprising a temperature sensor configured to measure the temperature of the power source and communicate the measured temperature to the electronic protection circuitry.

6. The negative pressure wound therapy system of claim 1, wherein the electronic protection circuitry is configured to:
monitor a current provided by the power source;
responsive to the temperature satisfying the at least one temperature threshold and the current not increasing over a duration of time, cause the electronic control circuitry to deactivate the negative pressure source; and
responsive to the temperature satisfying the at least one temperature threshold and the current increasing over the duration of time, prevent supply of power from the power source.

7. The negative pressure wound therapy system of claim 6, wherein the at least one temperature threshold comprises first and second temperature thresholds, the second temperature threshold being associated with temperature that is higher than temperature associated with the first temperature threshold, and wherein the integrated circuit is configured to:
responsive to the temperature satisfying the first temperature threshold and the current not increasing over the duration of time, cause the electronic control circuitry to decrease activity of the negative pressure source; and
responsive to the temperature satisfying the second temperature threshold and the current not increasing over the duration of time, cause the electronic control circuitry to deactivate the negative pressure source.

8. The negative pressure wound therapy system of claim 6, wherein the electronic protection circuitry is configured to:
responsive to the temperature not satisfying the at least one temperature threshold and the current increasing over the duration of time, cause the electronic control circuitry to deactivate the negative pressure source.

9. The negative pressure wound therapy system of claim 6, wherein the integrated circuit is configured to determine that the current is increasing responsive to the current satisfying a maximum current threshold.

10. The negative pressure wound therapy system of claim 9, wherein the maximum current threshold comprises 320 mA.

11. A negative pressure wound therapy system comprising:
a wound dressing configured to be placed over a wound, the wound dressing configured to absorb fluid aspirated from the wound;
a plurality of electronic components disposed on or within the wound dressing and comprising:
a negative pressure source disposed on or within the wound dressing, the negative pressure source configured to aspirate fluid from the wound; and
a power source configured to provide power to the negative pressure source; and
a temperature protection system disposed on or within the wound dressing and comprising:
first and second substances enclosed in a pouch; and
a trigger configured to facilitate at least one of mixing of or reaction between the first and second substances responsive to a temperature of at least one electronic component of the plurality of electronic components satisfying a temperature threshold indicative of excessive temperature and trigger an endothermic reaction that limits an amount of heat generated by the at least one electronic component.

12. The negative pressure wound therapy system of claim 11, wherein the trigger comprises a bimetallic switch.

13. The negative pressure wound therapy system of claim 12, wherein the bimetallic switch comprises a bimetallic disk configured to change shape responsive to a temperature of the bimetallic disk satisfying the temperature threshold.

14. The negative pressure wound therapy system of claim 11, wherein the pouch is positioned between the at least one electronic component and the wound.

15. The negative pressure wound therapy system of claim 11, wherein the pouch at least partially surrounds the at least one electronic component.

16. The negative pressure wound therapy system of claim 11, wherein the at least one electronic component comprises the power source or the negative pressure source.

17. (canceled)

18. The negative pressure wound therapy system of claim 11, wherein the first and second substances are separated from one another.

19. The negative pressure wound therapy system of claim 11, wherein the first substance is stored in a first section of the pouch and the second substance is stored in a second section of the pouch, and wherein the trigger is configured to cause a breach in a separation between the first and second sections responsive to the temperature of the at least one electronic component satisfying the temperature threshold.

20. The negative pressure wound therapy system of claim 11, wherein the first substance comprises a salt or alcohol and the second substance comprises water.

21. The negative pressure wound therapy system of claim 20, wherein the first substance comprises a urea salt, sodium chloride, or propylene glycol.

22. (canceled)

23. (canceled)
